(19) United States
(12) Patent Application Publication
(10) Pub. No.: US 2020/0049619 A1
(43) Pub. Date: Feb. 13, 2020

(54) POLARIZED LIGHT FILTER VISION SYSTEM TO DETECT LEVEL OF TEMPER IN GLASS

(71) Applicant: GM GLOBAL TECHNOLOGY OPERATIONS LLC, DETROIT, MI (US)

(72) Inventors: Jerod F. Long, New Boston, MI (US); Robert Denk, Bloomfield Hills, MI (US); Tyler Boscoe, Harrison Township, MI (US)

(21) Appl. No.: 16/057,883

(22) Filed: Aug. 8, 2018

(51) Int. Cl. G01N 21/21; C03B 27/00; G01N 33/38

(52) U.S. Cl. CPC G01N 21/21; G01N 2021/177; G01N 33/386; C03B 27/00

(57) ABSTRACT

A method to inspect for proper tempering of a piece of glass includes forming a piece of tempered glass and exposing the piece of tempered glass to a polarized light source. A vision system is used to inspect a temper pattern of the piece of tempered glass being exposed to the polarized light source and then the temper pattern of the piece of tempered glass is compared to a master temper pattern to determine if the inspected temper pattern is acceptable.

### FIELD

Apparatuses consistent with exemplary embodiments relate to product quality inspection methods. More particularly, apparatuses consistent with an exemplary embodiment relate to a polarized light filter vision system to detect level of temper in glass.

### BACKGROUND

Tempered glass is a type of safety glass processed by controlled thermal or chemical treatments to increase its strength compared with normal glass. Tempering puts the outer surfaces into compression and the interior into tension. Such stresses cause the glass, when broken, to crumble into small granular chunks instead of splintering into jagged shards as plate glass does. The granular chunks are less likely to cause injury.

As a result of its safety and strength, toughened glass is used in a variety of demanding applications, including passenger vehicle windows. Tempered glass is physically and thermally stronger than normal glass. The greater contraction of the inner layer during manufacturing induces compressive stresses in the surface of the glass balanced by tensile stresses in the body of the glass. It is this compressive stress that gives the tempered glass increased strength. Any cutting or grinding must be done prior to tempering. Cutting, grinding, and sharp impacts after tempering will cause the glass to fracture.

A strain pattern resulting from tempering can be observed with polarized light or by using a pair of polarizing sun glasses. However, this visual method of temper pattern detection is time consuming and thus may only be performed on an infrequent basis such that delays in production are minimized. Also, this infrequent sampling of temper patterns may compromise the level of confidence in compliance for all parts produced in contrast to inspecting a larger population of product.

### SUMMARY

One or more exemplary embodiments address the above issue by providing a method to inspect the level of temper of a piece of glass.

According to aspects of an exemplary embodiment, a method to inspect the level of temper of a piece of glass includes forming a piece of tempered glass and exposing the piece of tempered glass to a polarized light source. Other aspects as according to the exemplary embodiment include using a vision system to inspect a temper pattern of the piece of tempered glass being exposed to the polarized light source, and comparing the temper pattern of the piece of tempered glass with a master temper pattern to determine if the inspected temper pattern is acceptable.

And a further aspect of the exemplary embodiment includes processing the image of the inspected temper pattern using an image processor before comparing to the master temper pattern. Still further aspects of the exemplary embodiment include automatically marking scrapping the piece of tempered glass when the inspected temper pattern is determined to be unacceptable. And another aspect includes cooling the piece of tempered glass before exposing to polarized light source. And yet other aspects of the method include establishing the master temper pattern by making polarized filter images of formed pieces of tempered glass, and storing the polarized filter images of the temper patterns that comply with regulatory requirements in the vision system.

Further, in accordance with aspects of the exemplary embodiment, a system for detecting a level of temper in a piece of tempered glass includes a light source configured to illuminate a piece of tempered glass, and a light filter configured to polarize the light from the light source illuminating the piece of tempered glass. Other aspects of the system include a camera configured to capture an image of the piece of tempered glass being illuminated by the light source, and an image comparison device operative to compare the captured image to a master temper pattern to determine if the level of temper in the piece of tempered glass is acceptable. Still another aspect is provided wherein the image comparison device is a computer. Yet other aspects include an automatic marking tool for marking the piece of tempered glass determined to be unacceptable, or an automatic scrapping tool for scrapping the piece of tempered glass determined to be unacceptable. And still another aspect includes an image processor configured to process the captured image from the camera.

### BRIEF DESCRIPTION OF THE DRAWINGS

The present exemplary embodiment will be better understood from the description as set forth hereinafter, with reference to the accompanying drawings, in which:

FIG. 1A is an illustration of a piece of tempered glass that exhibits a good temper pattern when exposed to polarized light in accordance with an exemplary embodiment;

FIG. 1B is an illustration of a piece of tempered glass that exhibits a bad temper pattern when exposed to polarized light in accordance with the exemplary embodiment

FIG. 2 is an illustration of a vision system used to inspect pieces of tempered glass to determine a level of temper in accordance with aspects of the exemplary embodiment; and

FIG. 3 is an illustration of an flow diagram for the method to inspect the level of temper of a piece of glass in accordance with aspects of the exemplary embodiment.

### DETAILED DESCRIPTION OF THE INVENTION

The following description is merely exemplary in nature and is not intended to limit the present disclosure, application, or uses thereof.

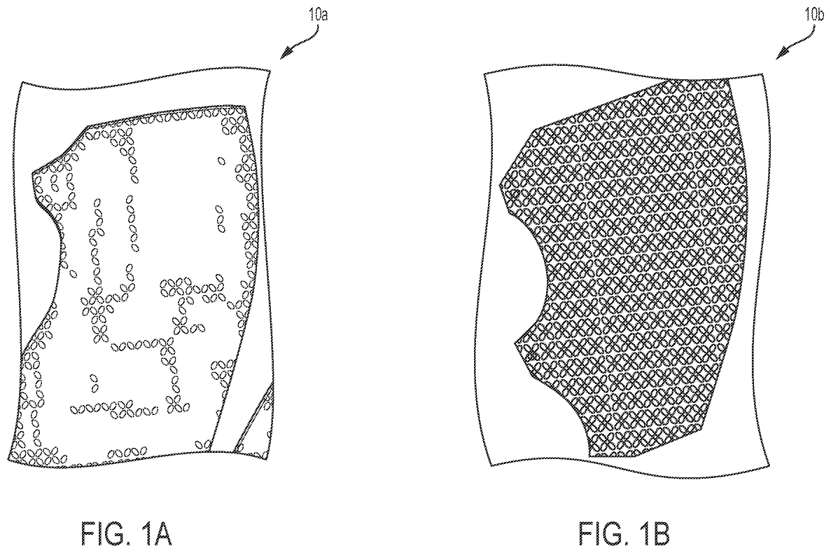

FIGS. 1A and 1B are illustrations of tempered glass that exhibits a good temper pattern 10a and a bad temper pattern 10b when the glass is exposed to polarized light in accordance with an exemplary embodiment. Tempered glass can be made from annealed glass via a thermal tempering process. The glass is placed onto a roller table, taking it through a furnace that heats it well above its transition temperature. The glass is then rapidly cooled with forced air drafts while the inner portion remains free to flow for a short time. As the center of the glass cools, it tries to pull back from the outer surfaces. As a result, the center remains in tension, and the outer surfaces go into compression, which gives tempered glass its strength.

In practice, there are slight differences in the rate of cooling across the glass, which results in slight differences in temper strength. These differences in temper strength show up optically when viewed under polarized light as a temper pattern which appear as a series of light and dark areas within the glass. Typically, if the temper pattern appears as blurry and light areas then the temper level is considered to be good. However, if the temper pattern appears as very clear and distinct areas then the temper level is considered to be bad.

Current methods for inspecting the proper tempering of glass involve visual inspection of the temper pattern within the glass or destructive testing, both of which are done by a person on a sample basis rather than every single piece of glass produced. This type of infrequent sampling reduces confidence of compliance with regulatory standards for all parts produced. The present invention seeks replace the existing visual inspection and destructive testing methods by a person on a sampling basis.

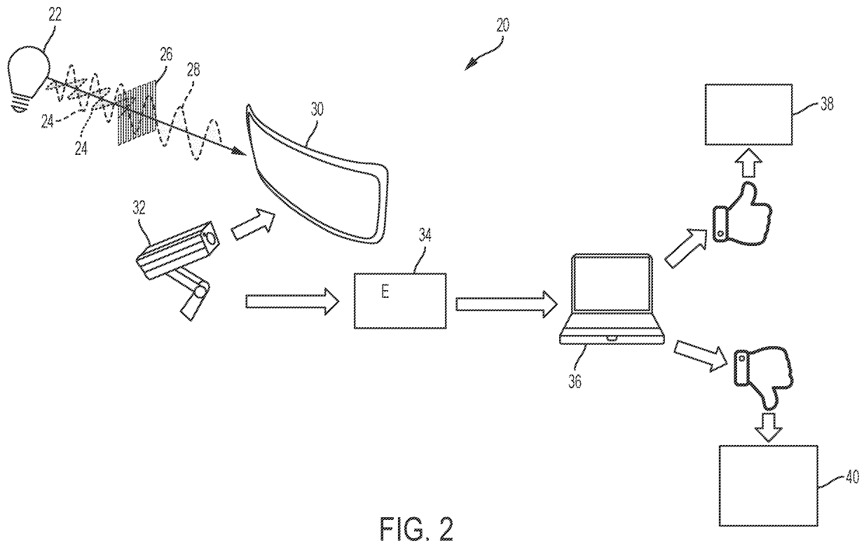

Referring now to FIG. 2 an illustration of high speed a vision system 20 used to inspect pieces of tempered glass to determine a level of temper in 100% of produced parts is provided in accordance with aspects of the exemplary embodiment. The vision system 20 includes a light source 22 that emits unpolarized light waves 24 through a light filter 26 that is configured to polarize the light waves 24 emitted from the light source 22. The polarized light wave 28 illuminated a piece of tempered glass 30. The polarized light wave 28 enhances the ability to detect differences in temper patterns that indicate whether a piece of glass 30 has been properly tempered or not.

A digital camera 32 is used to capture an image of the temper pattern of the piece of tempered glass 30 being illuminated by the filtered polarized light wave 28 emitted from the light source 22. The captured temper image from the camera 32 is received by an image processor 34 to process the captured temper image which is thereafter received by an image comparison device 36 operative to compare the captured temper image to a master temper pattern to determine if the level of temper in the piece of tempered glass 30 is acceptable.

The master temper pattern is established by making polarized filter images of formed pieces of tempered glass and storing the polarized filter images of the temper patterns within tempered glass that comply with regulatory requirements in the vision system. The image comparison device 36 may include the image processor 34 and can be a computer or similar device operative to compare the differences between two images and display the results.

If the image comparison device 36 determines that the captured temper image of a piece of tempered glass 30 exhibits a blurry and light pattern similar to the master temper pattern then the produced part is considered to be properly tempered and at 38 the piece of tempered glass 30 will continue to be processed. However, if the image comparison device 36 determines that the captured temper image of a piece of tempered glass 30 is very defined and in focus as compared to the master temper pattern then the produced part is considered to be improperly tempered and at 40 the piece of tempered glass 30 will be marked by an automatic marking tool as unacceptable, or the piece of tempered glass 30 will be scrapped by an automatic scrapping machine if determined to be unacceptable.

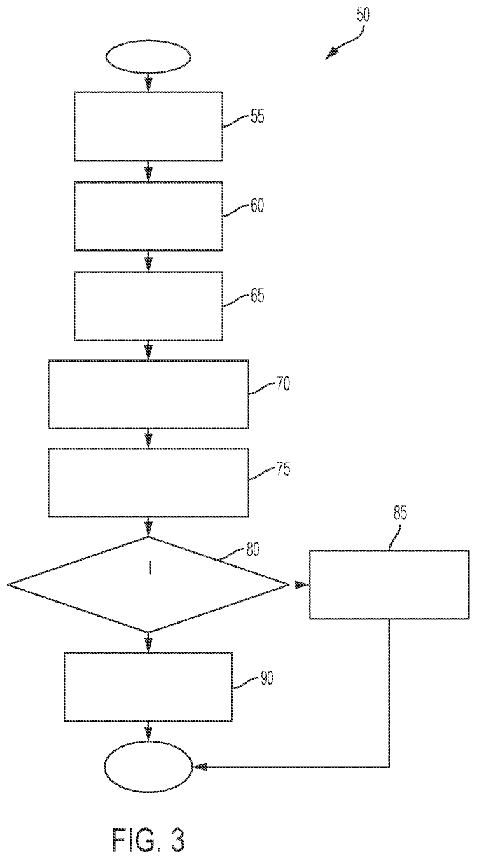

FIG. 3 is an illustration of a flow diagram 50 for the method to inspect the level of temper of a piece of glass in accordance with aspects of the exemplary embodiment. The method begins at block 55 with forming a piece of tempered glass and, at block 60, the piece of tempered glass undergoes a cooling process.

Next, at block 65 the method continues with exposing the piece of tempered glass polarized light to enhance the temper pattern within the glass. At block 70, the piece of tempered glass is inspected and an image of the temper pattern is captured by a camera. The method continues at block 75 where the captured temper image is processed through an image processor and then the image is compared to a master temper pattern.

At block 80, an image comparison device determines if the captured temper image is acceptable in comparison to the master temper pattern. If the captured temper image is determined to be acceptable then, at block 85, the piece of tempered glass will continue to be processed. If the captured temper image is determined to be unacceptable then, at block 90, the piece of tempered glass is automatically marked as unacceptable by an automatic marking tool, or automatically scrapped by an automatic scrapping machine.

The description of the invention is merely exemplary in nature and variations that do not depart from the gist of the invention are intended to be within the scope of the invention. Such variations are not to be regarded as a departure from the spirit and scope of the invention.

What is claimed is:

1. A method to inspect the level of temper of a piece of glass comprising:
forming a piece of tempered glass;
exposing the piece of tempered glass to a polarized light source;
using a vision system to inspect a temper pattern of the piece of tempered glass being exposed to the polarized light source; and
comparing the temper pattern of the piece of tempered glass with a master temper pattern to determine if the inspected temper pattern is acceptable.

2. The method of claim 1 further comprising processing the image of the inspected temper pattern using an image processor before comparing to the master temper pattern.

3. The method of claim 1 further comprising cooling the piece of tempered glass before exposing to polarized light source.

4. The method of claim 1 further comprising automatically marking the piece of tempered glass when the inspected temper pattern is determined to be unacceptable.

5. The method of claim 1 further comprising automatically scrapping the piece of tempered when the inspected temper pattern is determined to be unacceptable.

6. The method of claim 1 further comprising establishing the master temper pattern by making polarized filter images of formed pieces of tempered glass and storing the polarized filter images of the temper patterns that comply with regulatory requirements in the vision system.

7. A system for detecting a level of temper in glass comprising:
a light source configured to illuminate a piece of tempered glass;
a light filter configured to polarize the light from the light source illuminating the piece of tempered glass;
a camera configured to capture an image of the piece of tempered glass being illuminated by the light source; and
an image comparison device operative to compare the captured image to a master temper pattern to determine if the level of temper in the piece of tempered glass is acceptable.

8. The system of claim 7 wherein the image comparison device is a computer.

9. The system of claim 7 further comprising an image processor configured to process the captured image from the camera.

10. The system of claim 7 further comprising an automatic marking tool for marking the piece of tempered glass determined to be unacceptable.

11. The system of claim 7 further comprising an automatic scrapping machine for scrapping the piece of tempered glass determined to be unacceptable.
